(19) United States
(12) Patent Application Publication
(10) Pub. No.: US 2005/0106637 A1
(43) Pub. Date: May 19, 2005

(54) Methods for assessing muscle protein wasting and therapeutics therefor

(76) Inventor: Jie Du, League City, TX (US)

Correspondence Address: GREENLEE WINNER AND SULLIVAN P C, 4875 PEARL EAST CIRCLE, SUITE 200, BOULDER, CO, 80301, US

(21) Appl. No.: 10/818,908

(22) Filed: Apr. 5, 2004

Related U.S. Application Data
(63) Continuation of application No. 10/008,575, filed on Nov. 7, 2001, now abandoned.
(60) Provisional application No. 60/246,450, filed on Nov. 7, 2000.

(51) Int. Cl. G01N 33/53; G01N 33/567; G01N 33/557

(52) U.S. Cl. 435/7.2; 436/517

(57) ABSTRACT

The present disclosure provides evidence that actin degradation, in particular to produce a proteolytic fragment of about 14 kDa, is an important marker of muscle protein wasting. Muscle biopsy samples from a patient suffering from, susceptible to, or treated for a catabolic condition are tested for the production of an actin cross reacting protein of about 14 kDa, as estimated by SDS-PAGE. The presence of this actin fragment is diagnostic of muscle protein wasting (catabolism) in a muscle biopsy sample. Where a treatment for the catabolic disorder and/or muscle protein wasting is given, the failure to observe reductions in the amount of the actin degradative product is diagnostic of a treatment which is not effective to ameliorate or prevent the wasting. Animal models in which there is muscle protein wasting, e.g., streptozotocin-treated diabetic rates, can be used to identify inhibitors of wasting by reduction in actin fragments in muscle biopsy specimens of treated animals. Inhibitors identified in an animal model (or a cell culture model) can be used to prevent or decrease muscle wasting in a patient with a catabolic illness or used to increase muscle mass in a normal human or animal, an animal or human recovering from a catabolic illness or in a farm animal.

### CROSS REFERENCE TO RELATED APPLICATIONS

This application is a continuation of U.S. patent application Ser. No. 10/008,575, filed Nov. 7, 2001, which application claims benefit of U.S. Provisional Application No. 60/246,450, filed Nov. 7, 2000.

### ACKNOWLEDGMENT OF FEDERAL RESEARCH SUPPORT

This invention was made, at least in part, with funding from the National Institutes of Health (Grant NO. DK 37175). Accordingly, the United States Government has certain rights in this invention.

### BACKGROUND OF THE INVENTION

The field of this invention is medicine, and in particular, this invention relates to assays for muscle protein degradation (wasting), to assays to monitor the efficacy of a treatment for wasting in patients and to the use of assays for actin degradation products.

Because of the detrimental effects of wasting, especially of muscle, which is associated with variety of catabolic disorders, there is a need in the art to rapidly diagnose wasting in a patient before the damage is extensive and debilitating so that appropriate treatments can be administered. There is also a longfelt need in the art for ways to identify therapeutic compositions which prevent or reduce the degradation of muscle proteins associated with wasting. The present invention meets these needs.

### SUMMARY OF THE INVENTION

The present invention provides methods for the diagnosis of muscle protein wasting and for monitoring the efficacy of treatments for inhibition or prevention of muscle protein wasting in a patient suffering from or susceptible to muscle protein wasting. Specifically, the present inventors have identified a particular actin breakdown product (of about 14-15 kDa) as characteristic of the CASPASE 3-mediated breakdown of muscle protein associated with wasting.

In diagnostic or treatment monitoring methods, a muscle biopsy specimen is obtained from the patient, homogenized, the proteins are reduced and size-separated using sodium dodecyl sulfate-polyacrylamide gel electrophoresis, and the actin-related proteins are detected using immunoblotting techniques. Desirably the specimen (sample) is from about 5 to about 500 mg, typically about 50 mg. A protein of about 14-15 kDa which cross reacts with antibody specific for actin is a marker of muscle protein wasting. When a patient being tested for muscle protein wasting displays the actin-related degradation product of about 14-15 kDa in muscle biopsy tissue, then that patient is deemed to be undergoing muscle protein wasting.

If a patient who has been exhibiting symptoms of protein wasting is responding to a treatment designed to inhibiting the wasting, the 14-15 kDa actin degradation product in the muscle biopsy tissue is reduced or is no longer present.

Animal models for muscle protein wasting can be treated with inhibitors of muscle protein wasting (caspase 3 inhibitors). Suitable animal models include, but are not limited to, rats in which diabetes has been induced by streptozotocin. The present invention further provides a method for increasing muscle mass or preventing loss of muscle mass in a subject achieved by administering to the subject an effective amount of a caspase enzyme inhibitor or an inhibitor of an activator of caspase enzyme or of the enzymes that activate caspases (e.g., enzymes or chemicals that block the activity of phosphatidylinositol 3-kinase). Muscle mass is increased in a normal human or animal, in a human or animal recovering from a muscle wasting condition or in a patient with a catabolic condition. The animal can be a farm animal. Alternatively, inhibitors of specific caspase enzymes (e.g. caspase 3) or inhibitors of the activation of caspase enzymes (e.g., a dominant negative gene or myoblasts transfected to express an inhibitor of caspases) can be introduced locally into muscle. Likewise, a “muscle-specific” gene could be used to avoid a generalized suppression of apoptosis events in other tissues/organs in animals and in the future, patients. A reduction in the amount of the actin degradation product in a muscle biopsy sample is characteristic of a positive response to the treatment. Similar studies can be carried out with muscle cells in culture, with actin proteins and degradation products being resolved using size separation techniques and identified with an actin-specific antibody.

### BRIEF DESCRIPTION OF THE DRAWINGS

FIG. 1 shows the induction of apoptosis as measured using DNA fragmentation with increasing dosage of staurosporine. DNA fragmentation was analyzed 24 hours after the addition of staurosporine to growing L6 muscle cells.

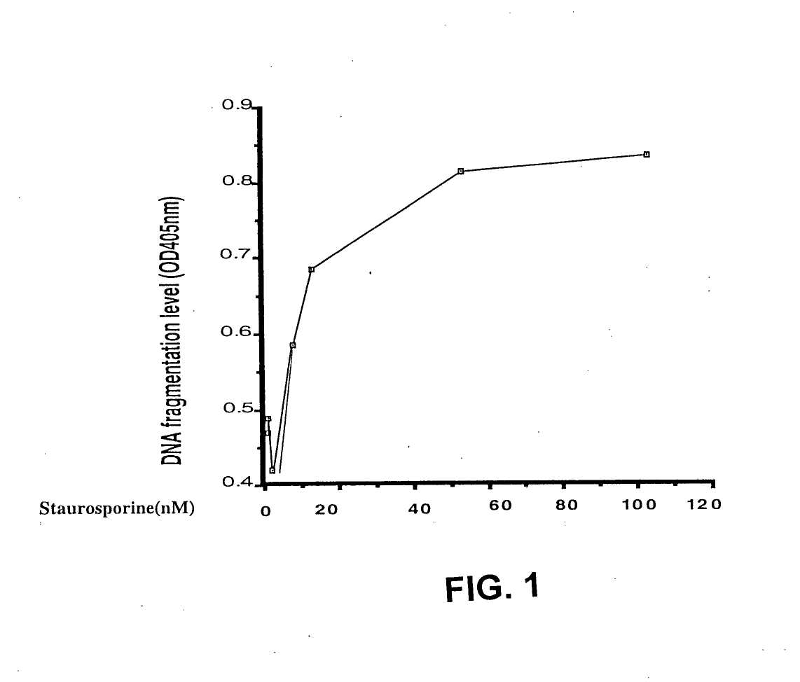

FIG. 2 shows the time-dependent cleavage of actin in lysates incubated for 0-3 hours at 37° C. Lysates were prepared from apoptotic L6 muscle cells (induced for 24 hours with 10 nM staurosporine). Lysate proteins were reduced and solubilized and separated by SDS-PAGE. Actin-related proteins were visualized by Western blotting using antibody specific for the C-terminal 11 amino acids of actin. The positions of the 14 kDa and 42 kDa protein bands are marked.

FIG. 3 demonstrates that staurosporine added to growing L6 muscle cells triggers cleavage of actin to yield a 14 kDa degradation product which cross-reacts with actin-specific antibody. The increase in the amount of the 14 kDa protein with increasing dosage of staurosporine is obvious in this image. Proteins were analyzed after 3 hours of incubation at 37° C., with lysates prepared from cells treated for 24 hours with 0-50 nM staurosporine.

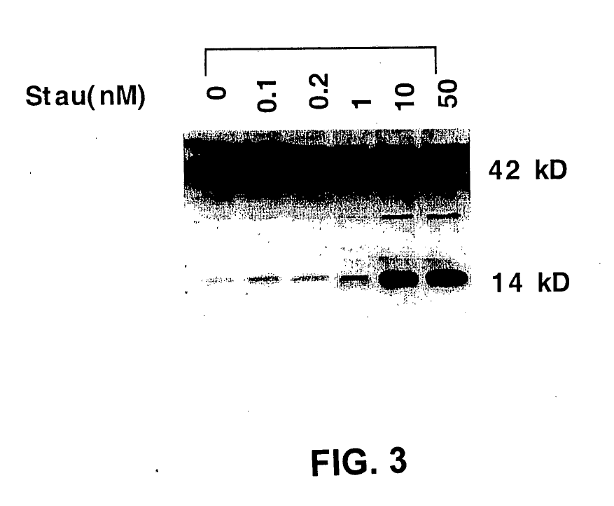

FIG. 4 shows the activation of caspase 3 with increasing concentrations of staurosporine added to growing L6 muscle cells and incubated for 3 hours. The caspase-specific proteins were separated by SDS-PAGE and visualized by immunoblotting with polyclonal antibodies prepared in response to the N-terminus of caspase 3.

FIG. 5 illustrates that actin cleavage is mediated by caspase 3. Treatment of the L6 muscle cells with staurosporine and/or the caspase inhibitor Ac-DEVD-CHO is shown.

FIG. 6 shows the effects of ATP, staurosporine, temperature and the proteasome inhibitor MG132 on actin degradation. Lysates are prepared and the proteins in the cell lysates incubated for 3 hours at 37° C.; lysates were prepared from L6 cells treated with 10 nM staurosporine for 24 hours.

FIG. 7 shows the effects of the proteasome inhibitor MG132 (20 μM) on cells treated with staurosporine (10 nM, 24 hours) or on control cells. Actin cleavage was measured in lysates immediately after lysis.

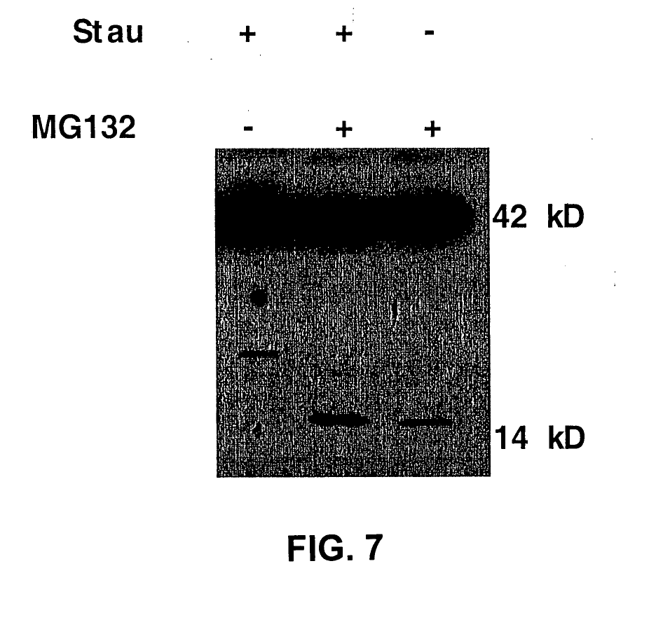

FIG. 8 demonstrates that the degradation of the 14 kDa actin degradation fragment involves the N-end rule pathway. Extracts of staurosporine-induced cells were incubated with the dipeptide inhibitor of E3α, Arg-Ala (2 mM) or 2 mM ATP during incubation at 37° C. for 3 hours.

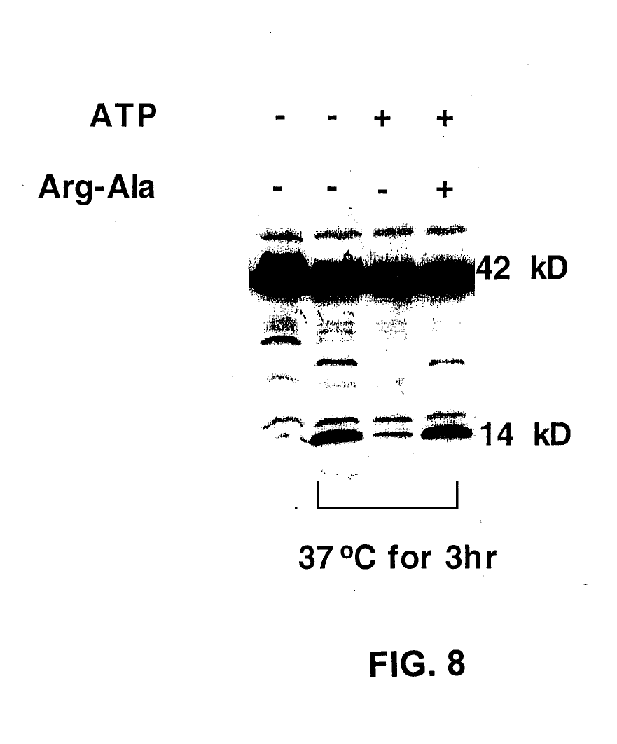

FIG. 9 demonstrates that overall protein degradation is increased in staurosporine-treated L6 muscle cells. L6 muscle cells were treated as marked, and lysates were incubated at 37° C. for 3 hours in the presence or absence of ATP (2 mM). Degradation of endogenous protein was monitored by measuring release of acid-soluble tyrosine from cell protein.

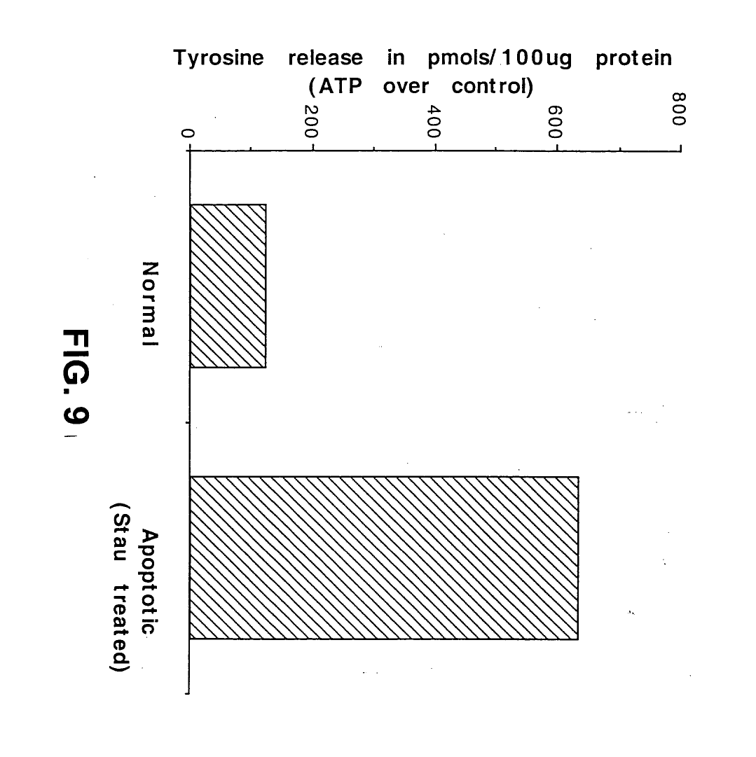

FIG. 10 demonstrates that the actin fragment is present in a model of accelerated degradation of muscle proteins, namely, streptozotocin-induced diabetes. Muscles from diabetic and sham-injected, pair-fed rats were harvested, homogenized and tested for the presence of the actin fragment. There was more than a two-fold increase in the amount of the 14 kDa actin fragment in muscles of diabetic rats. Similar findings were obtained in another rat model of a catabolic condition (chronic renal insufficiency) compared to sham-operated, pair-fed rats.

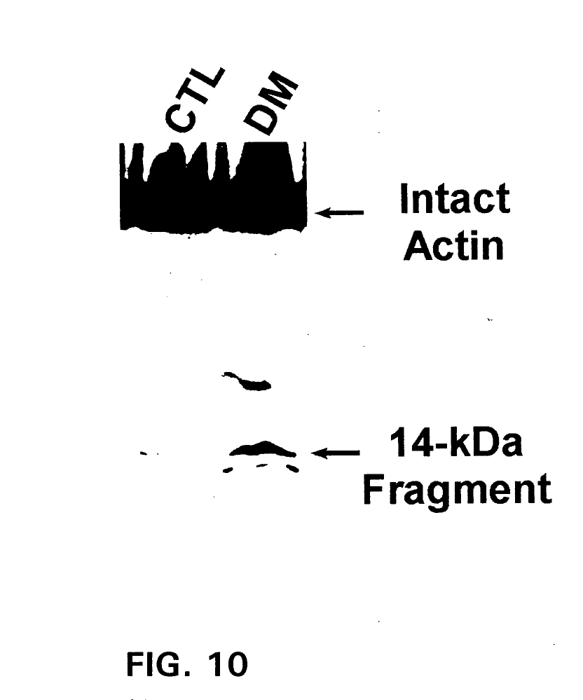

### DETAILED DESCRIPTION OF THE INVENTION

Loss of lean body mass is a major complication of many catabolic conditions including, but not limited to, uremia, sepsis, cancer, metabolic acidosis and diabetes. Studies of rats and humans with catabolic conditions show that the primary proteolytic pathway stimulated in muscle is the ubiquitin-proteasome pathway.

Purified monomeric myofibril proteins are degraded rapidly by the ubiquitin-proteasome pathway. However, intact myofibrils or soluble actomyosin protein complexes are resistant to degradation buy this pathway. These finding indicate that an important step in the proteolysis of the myofibrillar proteins is the dissociation of free myosin, actin and/or other contractile proteins.

Signals that activate muscle proteolysis in catabolic conditions could be used to identify the initial step(s) that result in breakdown of muscle proteins. Diabetes activates muscle proteolysis via the ubiquitin-proteasome system and insulin, a well-known inhibitor of apoptosis, rapidly reverses this response. Moreover, chronic renal failure, sepsis, cancer, burn injury, etc. are associated with higher levels of cytokines and cytokines can trigger apoptosis pathways including activation of caspases, a family of aspartic acid proteases (17). For these reasons, we examined whether signals that activate caspase activity in muscle cells could initiate actomyosin dissociation and degradation of protein.

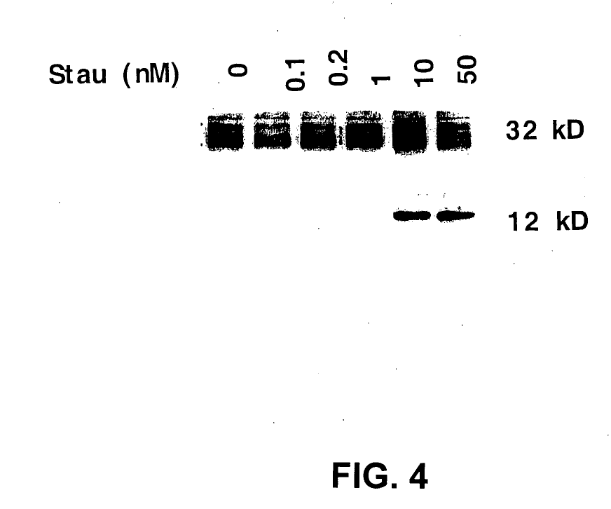

We found that staurosporine activates apoptosis in cultured L6 muscle cells as determined by the Death ELISA assay (commercially available from Boehringer Mannheim). The induction of apoptosis activates aspartic acid proteases, and we found that staurosporine does induces caspase 3 activity in L6 cells (FIG. 4) To determine if caspase 3 can cleave actomyosin, we added recombinant, active caspase 3 to isolated pure actomyosin complexes. Subsequently, proteins were separated and subjected to Western blot analysis using affinity-purified, polyclonal antibodies against the carboxy-terminus of actin (Sigma St. Louis, Mo.). There was a major ˜42-kDa band that corresponds to the expected size of monomeric rat skeletal muscle actin (Mr=41,816). In addition, there was a second ˜14 kDa band representing an actin fragment of approximately 14 kDa. These results indicate that caspase 3 can cleave actomyosin complexes and actin resulting in fragments of the constituent proteins.

Next, we incubated L6 muscle cells with 50 nM staurosporine for 3 h and then subjected them to lysis, and the resulting cell extracts were incubated at 37° C. for 3 to measure overall protein degradation as the release of tyrosine (18,19). With this method, protein degradation in extracts of control cells was slightly less than that in extracts from staurosporine-treated cells.

However, the 3 hour incubation at 37° C. could limit ATP and impair activity of the ubiquitin-proteasome system. When we added exogenous ATP to the extracts, tyrosine release by extracts from staurosporine-treated cells was significantly higher than by control cell extracts. These results indicate that stimulation of apoptotic pathways accelerate protein degradation in muscle cells.

Since caspase 3 is activated by induction of apoptotic pathways and can cleave actomyosin complexes, we expect that fragments of actin are generated in muscle cells stimulated to activate apoptosis. To test this possibility, L6 cells were treated with 50 nM staurosporine in 2% horse serum for 3 h. The cell extracts were incubated at 37° C. for various times before proteins were separated and subjected to Western blot analysis using commercially available, affinity-purified, polyclonal antibodies directed against the carboxy-terminus of actin. Again, there was a major ˜42 kDa band that corresponds to the expected size of monomeric rat skeletal muscle actin (Mr=41,816) in extracts incubated at 37° C. Incubation for 90 minutes resulted in the appearance of a second ˜14 kDa band; the intensity of this band increased with longer incubation times.

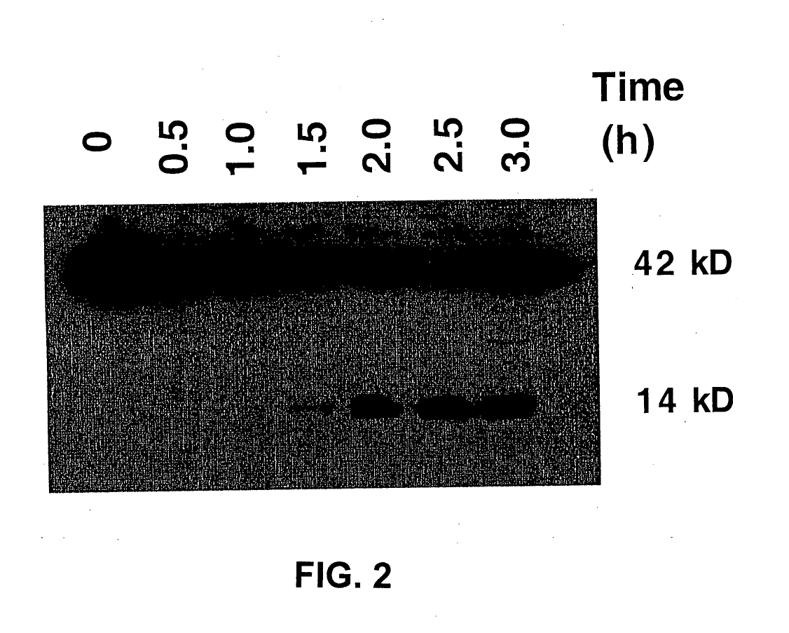

The appearance of the ˜14 kDa actin fragment was dependent upon the amount of staurosporine added to the cells (FIG. 2) with 100 pM staurosporine, the cleaved 14 kDa actin fragment was detected in cell extracts and its amount increased with higher concentrations of staurosporine. The 14 kDa actin fragment was absent in extracts from L6 cells kept on ice following treatment with 50 nM staurosporine; thus, we have concluded that the appearance of this fragment is the result of enzymatic action.

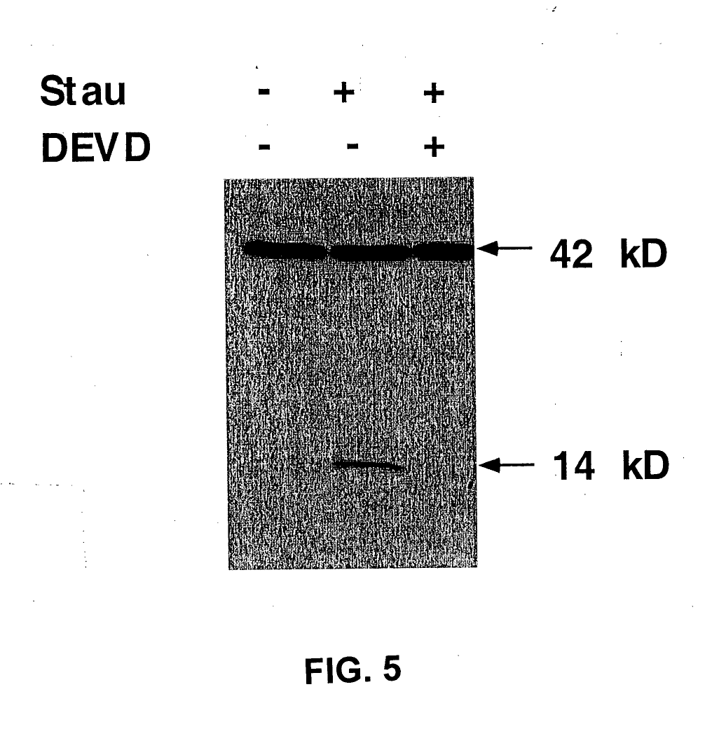

Stimulation of the apoptotic pathways results in a proteolytic cascade with activation of several isoforms of caspase. Since we found that staurosporine induces caspase 3 activity in L6 cells, we examined whether caspase 3 is involved in the appearance of the 14 kDa actin fragment using an inhibitor of caspase 3. The inhibitor was designed from the amino acid recognition sequence of caspase 3, DEVD, and a cell-permeable inhibitor derivative, Ac-DEVD-Cho, has been developed (20,21). To induce caspase 3 activity, L6 muscle cells were treated with 10 nM staurosporine or serum starvation, and cell extracts were prepared. The extracts were incubated with or without 5 μM Ac-DEVD-Cho for 3 hr and then tested for the appearance of the 14 kDa actin fragment: Ac-DEVD-Cho blocked the appearance of the actin fragment (FIG. 5). This finding indicates that activation of caspase 3 can be an initial step in the proteolytic processing of actin. To support this conclusion, we incubated lysates of L6 muscle cells at 37° C. with recombinant caspase 3 and then used Western blotting and detected the appearance of the actin fragment. The 14 kDa actin fragment was present as it was when we added recombinant, active caspase 3 to isolated pure actomyosin complexes.

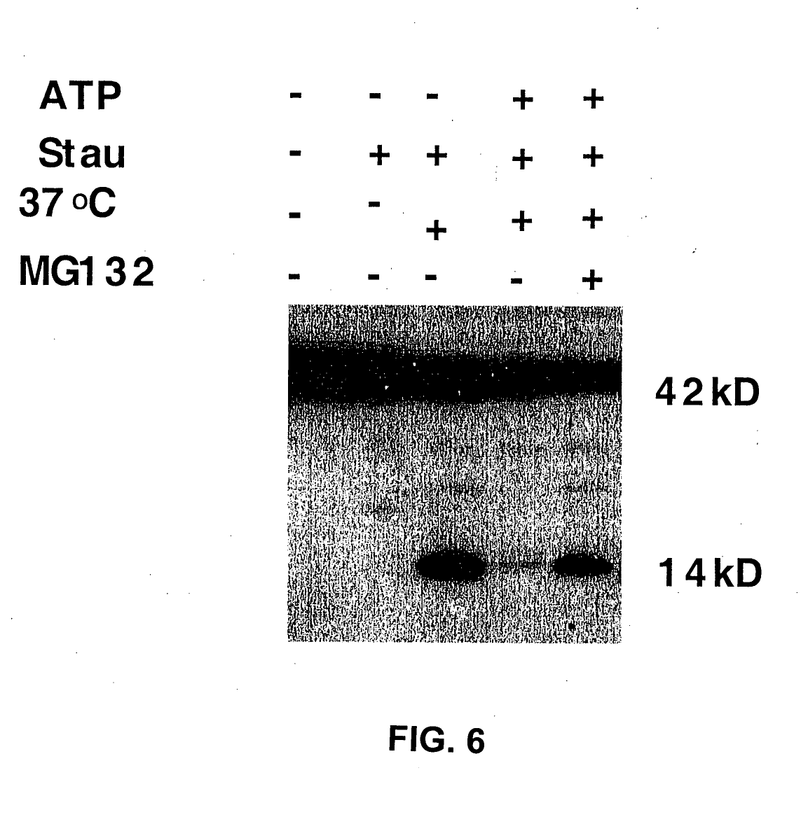

Interestingly, the 14 kDa actin fragment was not detected in cell lysates that had been incubated for short periods even though we found that caspase 3 is rapidly activated by incubation with staurosporine. Previously, Sun et al. noted that actin fragments are not detected when apoptotic pathways are stimulated in muscle cells. One possible explanation for this result could be a rapid degradation of the actin fragments. For example, ATP could become rate-limiting with prolonged incubations resulting in accumulation of the actin fragments. In this case, adding ATP to cell lysates during the 37° C. incubation would eliminate the actin fragments. To test this possibility, 2 mM ATP was added to extracts from staurosporine-treated cells at the start of the 3 hr, 37° C. incubation. As seen in FIG. 6, the amount of the 14 kDa peptide present was significantly reduced compared to the amount present when the same extract was incubated without added ATP. To test for involvement of the proteasome, we added MG132, an inhibitor of proteasome activity (ProScript, Inc. Cambridge, Mass.), and ATP to cell extracts before the 3 hr incubation at 37° C. Adding MG132 resulted in greater amounts of the 14 kDa actin peptide, despite the presence of ATP (FIG. 6).

These in vitro results are consistent with a proposed sequence of events beginning with apoptotic signals that generate active caspase 3, which cleaves actomyosin complexes and actin to yield the carboxy-terminal fragment of actin. This fragment (and others) are then rapidly degraded by the ubiquitin-proteasome system. To determine if similar events occur in intact cells, L6 cells were incubated for 24 hr with staurosporine in the presence or absence of 100 mM MG 132; control cells were treated with MG132 alone. The cells were then lysed and tested for the presence of the 14 kDa actin peptide. There was no detectable 14 kDa actin fragment in L6 cells that had been treated with staurosporine alone and there was only a small amount of the 14 kDa actin fragment in cells treated with MG132 alone. However, in L6 cells activated with staurosporine but treated with the proteasome inhibitor, the actin peptide was readily detected (FIG. 5).

Recently, it was reported that degradation of muscle proteins by the ubiquitin-proteasome system in catabolic conditions involves a combination of a specific E2 ubiquitin-carrier protein and E3 ubiquitin-protein ligase and involved the N-end rule pathway (22,23). The strategy involved blocking proteolysis with the dipeptide, Lys-Ala, which inhibits E3 ligase activity. To determine how activation of caspase 3 influences the N-end rule pathway, L6 cell extracts were prepared following exposure to staurosporine and incubated at 37° C. with or without the dipeptide Arg-Ala and ATP. As with the results of adding the proteasome inhibitor, MG132, the presence of this inhibitor of E3α activity resulted in a greater amount of the 14 kDa actin fragment compared to cells incubated with ATP alone. These results indicate that the N-end rule pathway is involved in degradation of the 14 kDa actin fragment.

Physiological conditions trigger caspase 3-mediated actin cleavage . To examine whether a catabolic condition that is known to accelerate protein degradation in muscle cells also involves actin cleavage, L6 muscle cells were activated by 24 h of serum starvation (0.5% FBS) and cell lysates were prepared. Serum starvation resulted in actin cleavage. This actin cleavage is caspase 3-dependent because adding Ac-DEVD-Cho to the cell lysate during the incubation blocked the accumulation of the 14 kDa actin fragment. Additional evidence for involvement of apoptosis-associated pathways was obtained by activating L6 cells using the serum starvation procedure and then adding insulin or IGF I because either hormone can prevent serum starvation induced apoptosis (24,25). Addition of 10 nM insulin or IGF I effectively blocked serum starvation-induced actin cleavage.

To determine if these results obtained in cultured cells also occur in intact animals, we examined the muscle of rats with acute diabetes, a condition that causes loss of muscle mass by accelerated protein degradation via activation of the ubiquitin-proteasome system (4,26). Rats were treated with streptozotocin and compared to pair-fed, rats injected with saline as described (4,26). Crude muscle lysates were isolated from acutely diabetic and control rats and examined for the presence of the 14 kDa actin fragment. A significant amount of this actin fragment was present in the muscle of diabetic rats compared to results from control rats. Finally, epitrochlearis muscles were incubated in Krebs-Henseleit bicarbonate buffer with or without the addition of the cell-permeable inhibitor of caspase 3, Ac-DEVD-Cho. Incubation with this inhibitor reduced the rate of muscle protein degradation by 30%.

To determine if a condition that activates protein degradation in muscle also activates actin degradation, we studied rats with streptozotocin-induced diabetes (4,26) and their sham-injected, pair-fed controls. This diabetic animal model exhibits accelerated loss of muscle mass and protein degradation in muscle that is mediated by the ubiquitin-proteasome pathway, and there was a more than two-fold increase in the actin fragment in muscle of the diabetic rats. This response is consistent with the results showing that caspase 3-induced degradation of actin leads to its degradation by the ubiquitin-proteasome system.

Monoclonal or polyclonal antibodies, preferably monoclonal, specifically reacting with a protein of interest can be made by methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience/Greene Publishing, New York, N.Y.

Antibodies specific for actin may be useful, for example, as probes for screening DNA expression libraries or for detecting the presence of actin or cross-reactive protein of 14-15 kDa in a test sample. Frequently, the polypeptides and antibodies will be labeled by joining, either covalently or noncovalently, a substance which provides a detectable signal. Suitable labels include but are not limited to radionuclides, enzymes (including but not limited to, alkaline phosphatase and horse radish peroxidase), substrates, cofactors, inhibitors, fluorescent agents, chemiluminescent agents, magnetic particles and the like. United States Patents describing the use of such labels include but are not limited to U.S. Pat. Nos. 3,817,837; 3,850,752; 3,939,350; 3,996,345; 4,277,437; 4,275,149; and 4,366,241.

Antibodies specific for an actin degradation product, especially a C-terminus degradation product, are useful identifying proteolytic breakdown products of actin resulting from the activation of caspase 3. Such antibodies can be obtained by the methods well known to the art or they can be purchased from commercial suppliers, for example, Sigma Chemical Co., St. Louis, Mo.

The discussion of the invention given herein is provided for illustrative purposes, and is not intended to limit the scope of the invention as claimed herein. Any variations in the exemplified articles which occur to the skilled artisan are intended to fall within the scope of the present invention.

All references cited herein are incorporated in the present application to the extent that there is no inconsistency with the present disclosure.

### EXAMPLES

### Example 1

### Cell Culture and Culture Conditions

L6 skeletal muscle cells (American Type Culture Collection, Manassas, Va.) were maintained in DMEM medium (1000 mg/l glucose) containing 10% het-inactivated fetal calf serum under humidified 5% CO₂-95% O₂ atmosphere at 37° C. Confluent cells were induced to undergo apoptosis by adding staurosporine to a final concentration of 10 rinM. Apoptosis was assessed with the Death-ELISA kit (Boehringer Mannheim, Indianapolis, Ind.).

### Example 2

### Cell Extract Preparation

L6 muscles cells were washed and collected in ice cold PBS. Cells were swelled in hypotonic buffer (5 mM Tris pH 8.0, 1 mM 2-mercaptoethanol, 1% glycerol, 1 mM EDTA, 1 mM EGTA containing 0.1 mM phenylmethanesulfonyl fluoride (PMSF), leupeptin (5 μg/ml) and aprotinin (5 μg/ml) on ice for 30 minutes, and the cells were then gently homogenized with twenty strokes of a dounce homogenizer, with the materials being kept on ice. Homogenates were centrifuged at 3500 rpm (about 2000×g) for 10 minutes to remove nuclei and cell debris.

### Example 3

### Assessment of Actin Degradation

To assess for proteolytic cleavage of actin, extracts (20 ug of total protein) were incubated in 20 μl reaction volumes containing 25 mM Hepes (pH 7.5), 5 mM EDTA, 5 mM DTT for 3 h at 37° C. Reactions were terminated by adding Laemmli sample buffer, proteins were separated by SDS-PAGE, and immunoblot detection of actin fragments was carried out with an anti-actin antibody (Sigma).

To examine for the activation of actin degradation in a model expressing high activity of muscle protein degradation linked to activation of the ubiquitin-proteasome pathway, we examined the muscle of rats with streptozotocin-induced diabetes and sham-injected, pair-fed, control rats (26). Gastrocnemius muscles were homogenized in the hypotonic buffer (5 mM Tris (pH 8.0), 1 mM 2-mercaptoethanol, 1% glycerol, 1 mM EDTA and 1 mM EGTA containing 0.1 mM phenylmethanesulfonyl-fluoride (PMSF), leupeptin (5 ug/ml), and aprotinin (5 ug/ml)) on ice using 20 strokes of a dounce homogenizer. The homogenates centrifuged at 3500 rpm for 10 min to remove nuclei and debris and the pellet was resuspended in Laemmli-sample buffer before boiling for 5 minutes. The samples were centrifuged at 15,000 rpm (about 10,000×g) and proteins in the supernatant were separated by SDS-PAGE before an immunoblot of actin fragments was carried out with an anti-actin antibody (Sigma).

### Example 4

### Immunodetection

After homogenate samples were reduced and solubilized in sample buffer, the proteins were size-separated using SDS-PAGE (14% polyacrylamide gels). The proteins were then transferred to ECL hybond membranes, and blots were incubated with anti-actin antibodies (1:500 dilution) in 5% milk in TTBS for 2 hours at 37° C. Bound primary antibodies were detected with goat anti-rabbit IgG antibody conjugated with HRRP and analyzed with ECL Detection Kit.

### Example 5

### Degradation of Endogenous Proteins

Protein degradation in cell extracts was assessed by measuring the net release of free tyrosine because muscles neither synthesizes nor degrades this amino acid (18,19). For 3 hours, 50 μg of cell extract proteins were incubated at 37° C. in 0.1 ml of reaction buffer (20 mM Tris (pH 7.6), 2 mM DTT, 10 mM MgCl₂, 100 mM KCl, 5% glycerol) with or without 2 mM ATP. Trichloroacetic acid (10% final concentration) was added and the amount of free tyrosine released from proteins was measured spectrofluorometrically. Alternatively, total cell protein can be labeled by growth in the presence of one or more radioactive amino acids, e.g., ¹⁴C-labeled or ³H-labeled, and solubilization of radioactive label can be measured with the use of liquid scintillation counting.

### BIBLIOGRAPHY

1. Mitch, W. E. and Goldberg, A. L. Mechanisms of muscle wasting: The role of the ubiquitin-proteasome system. N. Engl. J. Med. 335, 1897-1905 (1996).

2. Rock, K. L. et al. Inhibitors of the proteasome block the degradation of most cell proteins and the generation of peptides presented on MHC class 1 molecules. Cell 78, 761-771 (1994).

3. Bailey, J. L. et al. The acidosis of chronic renal failure activates muscle proteolysis in rats by augmenting transcription of genes encoding proteins of the ATP-dependent, ubiquitin-proteasome pathway. J. Clin. Invest. 97, 1447-1453 (1996).

4. Price, S. R. et al. Muscle wasting in insulinopenic rats results from activation of the ATP-dependent, ubiquitin-proteasome pathway by a mechanism including gene transcription. J. Clin. Invest. 98, 1703-1708 (1996).

5. Lecker, S. H., Solomon, V., Mitch, W. E. & Goldberg, A. L. Muscle proteinbreakdown and the critical role of the ubiquitin-proteasome pathway in normal and disease states. J. Nutr. 129, 227S-237S (1999).

6. Solomon, V. and Goldberg, A. L. Importance of the ATP-ubiquitin-proteasome pathway in degradation of soluble and myofibrillar proteins in rabbit muscle extracts. J. Biol. Chem. 271, 26690-26697 (1996).

7. Huang, J. and Forsberg, N. E. Role of calpain in skeletal-muscle protein degradation. Proc. Natl. Acad. Sci. USA 95, 12100-12105 (1998).

8. Waterhouse, N. J. et al. Calpain activation is upstream of caspases in radiation-induced apoptosis. Cell Death Differ. 5, 1051-1061 (1998).

9. Williams, A. B. et al. Sepsis stimulates release of myofilaments in skeletal muscle by a calcium-dependent mechanism. FASEB J 13, 1435-1443. 1999. Ref Type: Generic

10. Song, Q. et al. Resistance of actin to cleavage during apoptosis. Proc. Natl. Acad. Sci. 94, 157-162 (1997).

11. Mashima, T. et al. Actin cleavage by CPP-32/apopain during the development of apoptosis. Oncogene 14, 1007-1012 (1997).

12. May, R. C. et al. Metabolic acidosis stimulates protein degradation in rat muscle by a glucocorticoid-dependent mechanism. J. Clin. Invest. 77, 614-621 (1986).

13. May, R. C. et al. Glucocorticoids and acidosis stimulate protein and amino acid catabolism in vivo. Kidney Int. 49, 679-683 (1996).

14. Price, S. R. et al. Acidosis and glucocorticoids concomitantly increase ubiquitin and proteasome subunit mRNAs in rat muscle. Am. J. Physiol. 267, C955-C960 (1994).

15. Baracos, V. Et al. An in vitro preparation of the extensor digitorum communis muscle from the chick (Gallus domesticus) for studies of protein turnover. Comp Biochem Physiol A 92, 555-563 (1989).

16. Tiao, G. et al. Energy-ubiquitin-dependent muscle proteolysis during sepsis in rats is regulated by glucocorticoids. J. Clin. Invest. 97, 339-348 (1996).

17. Ashkenazi, A. and Dixit, V. M. Death receptors: signaling and modulation. Sci. 1308 (1998).

18. Isozaki, Y. et al. Protein degradation and increased mRNAs encoding proteins of the ubiquitin-proteasome proteolytic pathway in BC3H1 myocytes require an interaction between glucocorticoids and acidification. Proc. Natl. Acad. Sci. USA 93, 1967-1971 (1996).

19. Du, J. Et al. Glucocorticoids induce proteasome C3 subunit expression in L6 muscle cells by opposing the suppression of its transcription by NF-kB. J. Biol. Chem. 275, 19661-19666 (2000).

20. Nicholson, D. W. et al. Identification and inhibition of the ICE/CED-3 protease necessary for mammalian apoptosis. Nature 376, 37-43 (1995).

21. Thornberry, N. A. and Lazebnik, Y. Caspases: enemies within. Sci. 281, 1312-1316 (1998).

22. Solomon, V. et al. The N-end rule pathway catalyzes a major fraction of the protein degradation in skeletal muscle. J. Biol. Chem. 273, 25216-25222 (1998).

23. Solomon, V. et al. Rates of ubiquitin conjugation increase when muscles atrophy, largely through activation of the N-end rule pathway. PROC Natl Acad Sci. USA 95, 12602-12607 (1998).

24. Le Roith, D. Regulation of proliferation and apoptosis by the insulin-like growth factor I receptor. Growth Horm IGF Res 10, S12-S13 (2000).

25. Niesler, C. U. et al. IGF-I inhibits apoptosis induced by serum withdrawal, but potentiates TNF-alpha-induced apoptosis in 3T3-L1 preadipocytes. J. Endocrin. 167, 165-174 (2000).

26. Mitch, W. E. et al. Evaluation of signals activating ubiquitin-proteasome proteolysis in a model of muscle wasting. Amer. J. Physiol. 276, C1132-C1138 (1999).

1. A method for diagnosing muscle protein wasting in a patient suffering from or susceptible to muscle protein wasting, said method comprising the steps of:
(a) homogenizing a muscle biopsy sample and solubilizing the proteins therein;
(b) separating the proteins solubilized in step (a) according to size;
(c) contacting the proteins separated by size in step (b) with a ligand specific for at least one actin protein;
(d) detecting the binding of a ligand specific for at least one actin protein to at least one
actin protein among the separated proteins,
whereby muscle protein wasting is detected when an actin protein of about 14 kDa (as estimated by sodium dodecyl sulfate polyacrylamide gel electrophoresis) is detected in the proteins solubilized and separated from the muscle biopsy sample and wherein said actin protein of about 14 kDa is present in an increased amount in said patient biopsy sample as compared with a muscle biopsy sample from a normal individual.

2. The method of claim 1 wherein the proteins are separated according to size using sodium dodecyl sulfate polyacrylamide gel electrophoresis.

3. The method of claim 1 wherein the ligand in step (c) is an actin-specific antibody.

4. The method of claim 4 wherein the actin-specific antibody is specific for the C-terminus of actin and is an antibody produced by a first animal species.

5. The method of claim 4 wherein the detecting in step (d) is carried out using a second antibody produced in a second animal species, which second antibody is specific for an antibody produced by the first animal species.

6. The method of claim 5 wherein the second antibody has a detectable label.

7. The method of claim 6 wherein the detectable label is an enzyme.

8. The method of claim 1 wherein the patient is suffering from or is susceptible to a catabolic disorder.

9. The method of claim 8 wherein the catabolic disorder is sepsis, cancer, starvation, uremia, malnutrition, diabetes, burn injury, chronic renal failure, metabolic acidosis, acquired immunodeficiency syndrome, muscle denervation or trauma.

10. A method of preventing or reducing the degradation of muscle protein in a subject having a condition that stimulates muscle protein breakdown comprising the step of administering to the subject a pharmaceutically effective amount of a caspase enzyme inhibitor or an inhibitor of an activator of caspase enzymes.

11. The method of claim 10, wherein the condition is a catabolic illness.

12. The method of claim 10, wherein the condition is selected from the group consisting of uremia, sepsis, metabolic acidosis, burn injury, AIDS, cancer, muscle denervation, trauma and diabetes.

13. The method of claim 10, wherein the caspase enzyme inhibitor is a caspase 3 inhibitor.

14. The method of claim 10, wherein the subject is selected from a human and an animal.

15. A method of increasing muscle mass or preventing loss of muscle mass in a subject achieved by administering to the subject an effective amount of a caspase enzyme inhibitor or an inhibitor of an activator of caspase enzyme or of the enzymes that activate caspases.

16. The method of claim 15, wherein the subject is a farm animal.

17. The method of claim 15 wherein the inhibitor is PI3 Kinase or PI34P2.

18. The method of claim 15, wherein the activity of phosphatidylinositol 3-kinase is blocked.
